(19) United States
(12) Patent Application Publication
(10) Pub. No.: US 2025/0114404 A1
(43) Pub. Date: Apr. 10, 2025

(54) Treatment of Lower Back Pain and Disc Degenerative Disease using Inducible Pluripotent Stem Cell Derived Mesenchymal Stem Cells and T Regulatory Cells Utility

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/908,482

(22) Filed: Oct. 7, 2024

Related U.S. Application Data
(60) Provisional application No. 63/588,034, filed on Oct. 5, 2023.

(51) Int. Cl. A61K 35/28; A61K 39/00

(52) U.S. Cl. CPC A61K 35/28; A61K 40/10; A61K 40/31

(57) ABSTRACT

Disclosed are means and compositions of matter for treating degenerative disc disease and associated pain in part by stimulating enhanced local perfusion and reduction of inflammation. In one embodiment administration of inducible pluripotent stem cell derived mesenchymal stem cells is performed wherein said cells are optimized for migration and/or retention into perispinal environment. In some embodiments said mesenchymal stem cells are optimized for enhanced angiogenesis and/or suppression of inflammation. In another embodiment inducible pluripotent stem cell derived T regulatory cells are administered alone or with said mesenchymal stem cells in order to elicit enhanced perispinal perfusion while concurrently reducing inflammation.

### CROSS-REFERENCE TO RELATED APPLICATIONS

The present application claims benefit of U.S. Provisional Patent Application Ser. No. 63/588,034, filed on Oct. 5, 2023, titled “Treatment of Lower Back Pain and Disc Degenerative Disease using Inducible Pluripotent Stem Cell Derived Mesenchymal Stem Cells and T Regulatory Cells”, the contents of which are incorporated herein by reference in its entirety.

### FIELD OF THE INVENTION

The invention pertains to the field of therapeutic biologics, more importantly the invention pertains to reduction of back pain and stimulation of intravertebral disc regeneration, more specifically the invention pertains to utilization of pluripotent stem cell derived T regulatory cells and mesenchymal stem cells for reduction of lumbar disc associated pain and stimulation of regeneration.

### BACKGROUND OF THE INVENTION

Currently it is accepted that the medical treatment of degenerative disc disease and associated spine ailments is one of the costliest medical conditions with an estimated annual direct cost in the United States of 33 billion and total annual societal cost exceeding 100 billion dollars. Indeed, in one's lifetime most individuals will experience an episode of significant back and or neck pain. Although most individuals will improve with non-operative intervention, a significant percentage will go on to require costly surgery or other medical intervention.

The etiology of chronic back pain is multi-factorial, but in a significant proportion of patients, degenerative disc disease is the underlying cause. As an individual ages, the intervertebral disc looses water, begins to collapse, and can ultimately fail to adequately support adjacent vertebrae. As a result, the neural element can become compressed within the neural foramen as well as the central canal of the spine leading to painful back conditions. Additionally, discogenic back pain, perhaps a less well understood condition, can also lead to painful back conditions as a result of disc degeneration.

The process of intervertebral disc degeneration occurs in all of us as we age and its treatment in symptomatic patients has significant socioeconomic impact. Many studies have shown that notochordal cells, the precursors of the disc, are no longer present after age 10. During embryogenesis, notochordal cells are believed to be responsible for the formation of spine and intervertebral disc, as well as for maintenance and metabolic control of the nucleus pulposus (NP) further in life. The relationship between loss of notochordal cells with age and the onset of disc degeneration can perhaps best be understood by the changes in biomechanics of the discs as a consequence of proteoglycan loss. The proteoglycans are the hydrophilic moiety of the intervertebral disc. These molecules are uniquely structured to hold water and therefore provide the cushioning quality of the intervertebral disc. It has been shown in recent studies that notochordal cells produce 1.5 fold more proteoglycans and extracellular matrix than terminally differentiated chondrocytes. As the notochordal cells differentiate to chondrocytes in the NP, less water holding proteoglycan matrix is available. A cascade of events ensues resulting in disc degeneration, desiccation and collapse. Consequently, the annulus fibrosis (AF) begins to fissure and cracks, contributing to a vicious cycle of disc degeneration potentially resulting in chronic lower back pain.

The intervertebral disc is comprised of an external AF made up primarily of lamellar bands of type I collagen that surrounds a soft gelatinous central NP made up primarily of type II collagen and a proteoglycan matrix. The proteoglycan moiety is a highly hydrophilic molecule capable of holding significant amounts of water. The water holding capacity of the NP, held within the confines of the intact AF, gives the intervertebral disc its unique function, particularly that of providing a mobile compressible distraction between adjacent vertebral bodies, and thus providing the unique flexibility of the spinal column.

### SUMMARY OF THE INVENTION

Preferred methods include embodiments of treating lumbar ischemia associated disc degenerative disease comprising administration of a therapeutic cell with enhanced retention ability, wherein said enhanced retention ability allows localization and mediation of therapeutic activities to ischemic areas.

Preferred embodiments include methods wherein said therapeutic cell is derived from a pluripotent stem cell.

Preferred embodiments include methods wherein said pluripotent stem cell is capable of immortal cell growth without senescence.

Preferred embodiments include methods wherein said therapeutic cell is an inducible pluripotent stem cell.

Preferred embodiments include methods wherein said therapeutic cell is a mesenchymal stem cell derived from said inducible pluripotent stem cell.

Preferred embodiments include methods wherein said mesenchymal stem cell possesses some features of mesenchymal stem cells but is not a mesenchymal stem cell.

Preferred embodiments include methods wherein said cell possesses features of mesenchymal stem cells and features of endothelial cells.

Preferred embodiments include methods wherein said cell possesses features of mesenchymal stem cells and features of endothelial progenitor cells.

Preferred embodiments include methods wherein said cell possesses features of mesenchymal stem cells and features of neural progenitor cells.

Preferred embodiments include methods wherein said cell possesses features of mesenchymal stem cells and features of chondrocyte progenitor cells.

Preferred embodiments include methods wherein said cell possesses features of mesenchymal stem cells and features of nucleus pulposus cells.

Preferred embodiments include methods wherein said cell possesses features allowing for enhanced angiogenesis.

Preferred embodiments include methods wherein said cell is generated to produce enhanced levels of VEGF as compared to non-manipulated cells.

Preferred embodiments include methods wherein said cell is generated to produce enhanced levels of VEGF-C as compared to non-manipulated cells.

Preferred embodiments include methods wherein said cell is generated to produce enhanced levels of HGF as compared to non-manipulated cells.

Preferred embodiments include methods wherein said cell is generated to produce enhanced levels of angiopoietin as compared to non-manipulated cells.

Preferred embodiments include methods wherein said cell is generated to produce enhanced levels of IGF as compared to non-manipulated cells.

Preferred embodiments include methods wherein said cell is generated to produce enhanced levels of FGF-1 as compared to non-manipulated cells.

Preferred embodiments include methods wherein said cell is generated to produce enhanced levels of FGF-2 as compared to non-manipulated cells.

Preferred embodiments include methods wherein said cell is generated to produce enhanced levels of FGF-5 as compared to non-manipulated cells.

Preferred embodiments include methods wherein aid cell is generated to produce enhanced levels of HGF as compared to non-manipulated cells.

Preferred embodiments include methods wherein said cell is generated to produce enhanced levels of EGF as compared to non-manipulated cells.

Preferred embodiments include methods wherein said cell is generated to produce enhanced levels of GDF-11 as compared to non-manipulated cells.

Preferred embodiments include methods wherein said cell is generated to produce enhanced levels of GDF-15 as compared to non-manipulated cells.

Preferred embodiments include methods wherein said cell is generated to produce enhanced levels of SDF-1 as compared to non-manipulated cells.

Preferred embodiments include methods wherein said cell is generated to produce enhanced levels of IL-3 as compared to non-manipulated cells.

Preferred embodiments include methods wherein said cell is generated to produce enhanced levels of IL-1 receptor antagonist as compared to non-manipulated cells.

Preferred embodiments include methods wherein said cell is generated to produce enhanced levels of IL-8 as compared to non-manipulated cells.

Preferred embodiments include methods wherein said cell is generated to produce enhanced levels of IL-4 as compared to non-manipulated cells.

Preferred embodiments include methods wherein said cell is generated to produce enhanced levels of IL-13 as compared to non-manipulated cells.

Preferred embodiments include methods wherein said cell is generated to produce enhanced levels of IL-10 as compared to non-manipulated cells.

Preferred embodiments include methods wherein aid cell is generated to produce enhanced levels of VEGF-A as compared to non-manipulated cells.

Preferred embodiments include methods wherein said cell is generated to produce enhanced levels of heme-oxygenase I as compared to non-manipulated cells.

Preferred embodiments include methods wherein said cell is generated to produce enhanced levels of VEGF placental growth factor as compared to non-manipulated cells.

Preferred embodiments include methods wherein said cell is generated to produce enhanced levels of IL-20 as compared to non-manipulated cells.

Preferred embodiments include methods wherein said cell is generated to produce enhanced levels of IL-22 as compared to non-manipulated cells.

Preferred embodiments include methods wherein said cell is generated to produce enhanced levels of IL-35 as compared to non-manipulated cells.

Preferred embodiments include methods wherein said cell is generated to produce enhanced levels of IL-37 as compared to non-manipulated cells.

Preferred embodiments include methods wherein said cell is generated to produce enhanced levels of calretinulin as compared to non-manipulated cells.

Preferred embodiments include methods wherein said cell is generated to produce enhanced levels of CD47 as compared to non-manipulated cells.

Preferred embodiments include methods wherein said cell is generated to produce enhanced levels of SERP-1 as compared to non-manipulated cells.

Preferred embodiments include methods wherein said cell is generated to produce enhanced levels of thrombospondin as compared to non-manipulated cells.

Preferred embodiments include methods wherein said cell is generated to produce enhanced levels of endosialin as compared to non-manipulated cells.

Preferred embodiments include methods wherein said cell is generated to produce enhanced levels of arginase as compared to non-manipulated cells.

Preferred embodiments include methods wherein said cell is generated to produce enhanced levels of galectin-1 as compared to non-manipulated cells.

Preferred embodiments include methods wherein said cell is generated to produce enhanced levels of galectin-3 as compared to non-manipulated cells.

Preferred embodiments include methods wherein said cell is generated to produce enhanced levels of galectin-5 as compared to non-manipulated cells.

Preferred embodiments include methods wherein said cell is generated to produce enhanced levels of indolamine 2,3, dioxygenase as compared to non-manipulated cells.

Preferred embodiments include methods wherein said cell is generated to express enhanced levels of COX-1 as compared to non-manipulated cells.

Preferred embodiments include methods wherein said cell is generated to express enhanced levels of COX-2 as compared to non-manipulated cells.

Preferred embodiments include methods wherein enhanced angiogenic properties of said cells are endowed by exposure to hypoxia.

Preferred embodiments include methods wherein enhanced angiogenic properties of said cells are endowed by transfection with one or more angiogenesis stimulating genes.

Preferred embodiments include methods wherein said transfection is accomplished by use of a viral vector.

Preferred embodiments include methods wherein said transfection is accomplished by use of cell fusion.

Preferred embodiments include methods wherein said transfection is accomplished by use of a liposomal vector

Preferred embodiments include methods wherein said transfection is accomplished by use of exosomes.

Preferred embodiments include methods wherein said transfection is accomplished by use of electroporation.

Preferred embodiments include methods wherein said transfection is accomplished by use of a naked mRNA administration.

Preferred embodiments include methods wherein said transfection is accomplished by use of a naked plasmid administration.

Preferred embodiments include methods wherein hypoxia is administered at a sufficient oxygen tension and for a sufficient time period to enhance stimulation production of FGF-1 at a level of more than 2 ng/ml per 1 million cells.

Preferred embodiments include methods wherein hypoxia is administered at a sufficient oxygen tension and for a sufficient time period to enhance stimulation production of FGF-1 at a level of more than 10 ng/ml per 1 million cells.

Preferred embodiments include methods wherein hypoxia is administered at a sufficient oxygen tension and for a sufficient time period to enhance stimulation production of FGF-1 at a level of more than 100 ng/ml per 1 million cells.

Preferred embodiments include methods wherein hypoxia is administered at a sufficient oxygen tension and for a sufficient time period to enhance stimulation production of FGF-2 at a level of more than 1 ng/ml per 1 million cells.

Preferred embodiments include methods wherein hypoxia is administered at a sufficient oxygen tension and for a sufficient time period to enhance stimulation production of FGF-2 at a level of more than 5 ng/ml per 1 million cells.

Preferred embodiments include methods wherein hypoxia is administered at a sufficient oxygen tension and for a sufficient time period to enhance stimulation production of FGF-2 at a level of more than 10 ng/ml per 1 million cells.

Preferred embodiments include methods wherein hypoxia is administered at a sufficient oxygen tension and for a sufficient time period to enhance stimulation production of VEGF-A at a level of more than 100 pg/ml per 1 million cells.

Preferred embodiments include methods wherein hypoxia is administered at a sufficient oxygen tension and for a sufficient time period to enhance stimulation production of VEGF-A at a level of more than 100 pg/ml per 1 million cells.

Preferred embodiments include methods wherein hypoxia is administered at a sufficient oxygen tension and for a sufficient time period to enhance stimulation production of VEGF-A at a level of more than 400 pg/ml per 1 million cells.

Preferred embodiments include methods wherein hypoxia is administered at a sufficient oxygen tension and for a sufficient time period to enhance stimulation production of VEGF-C at a level of more than 1 ng/ml per 1 million cells.

Preferred embodiments include methods wherein hypoxia is administered at a sufficient oxygen tension and for a sufficient time period to enhance stimulation production of VEGF-C at a level of more than 5 ng/ml per 1 million cells.

Preferred embodiments include methods wherein hypoxia is administered at a sufficient oxygen tension and for a sufficient time period to enhance stimulation production of VEGF-C at a level of more than 10 ng/ml per 1 million cells.

Preferred embodiments include methods wherein hypoxia is administered at a sufficient oxygen tension and for a sufficient time period to enhance stimulation production of angiopoietin at a level of more than 50 pg/ml per 1 million cells.

Preferred embodiments include methods wherein hypoxia is administered at a sufficient oxygen tension and for a sufficient time period to enhance stimulation production of angiopoietin at a level of more than 500 pg/ml per 1 million cells.

Preferred embodiments include methods wherein hypoxia is administered at a sufficient oxygen tension and for a sufficient time period to enhance stimulation production of angiopoietin at a level of more than 1 ng/ml per 1 million cells.

Preferred embodiments include methods wherein enhanced angiogenic properties of said cells are endowed by exposure to lithium oxide.

Preferred embodiments include methods wherein enhanced angiogenic properties of said cells are endowed by exposure to cobalt chloride.

Preferred embodiments include methods wherein enhanced angiogenic properties of said cells are endowed by exposure to trichostatin A.

Preferred embodiments include methods wherein enhanced angiogenic properties of said cells are endowed by exposure to sulforaphane.

Preferred embodiments include methods wherein enhanced angiogenic properties of said cells are endowed by exposure to phenylbutyrate.

Preferred embodiments include methods wherein enhanced angiogenic properties of said cells are endowed by exposure to valproic acid.

Preferred embodiments include methods wherein enhanced angiogenic properties of said cells are endowed by exposure to a toll like receptor 3 agonist.

Preferred embodiments include methods wherein said toll like receptor 3 agonist is New Castle Disease Virus.

Preferred embodiments include methods wherein said New Castle Disease Virus is attenuated.

Preferred embodiments include methods wherein said toll like receptor 3 agonist is double stranded RNA.

Preferred embodiments include methods wherein said toll like receptor 3 agonist is Poly IC.

Preferred embodiments include methods wherein said toll like receptor 3 agonist is Poly ICIC.

Preferred embodiments include methods wherein said toll like receptor 3 agonist is Poly LC.

Preferred embodiments include methods wherein enhanced angiogenic properties of said cells are endowed by exposure to a toll like receptor 4 agonist.

Preferred embodiments include methods wherein said toll like receptor 4 agonist is hyaluronic acid.

Preferred embodiments include methods wherein said toll like receptor 4 agonist is low molecular weight hyaluronic acid.

Preferred embodiments include methods wherein said toll like receptor 4 agonist is p19 peptide.

Preferred embodiments include methods wherein said toll like receptor 4 agonist is HMGB1.

Preferred embodiments include methods wherein said toll like receptor 4 agonist is beta glucan.

Preferred embodiments include methods wherein said toll like receptor 4 agonist is lipopolysaccharide.

Preferred embodiments include methods wherein said toll like receptor 4 agonist is Natural Monophosphoryl Lipid A.

Preferred embodiments include methods wherein said toll like receptor 4 agonist is CXR527.

Preferred embodiments include methods wherein said toll like receptor 4 agonist is neutrophil extracellular traps.

Preferred embodiments include methods wherein said toll like receptor 4 agonist is free histones.

Preferred embodiments include methods wherein said toll like receptor 4 agonist is FP11-18.

Preferred embodiments include methods wherein said toll like receptor 4 agonist is FP111.

Preferred embodiments include methods wherein said toll like receptor 4 agonist is FP20-24.

Preferred embodiments include methods wherein said toll like receptor 4 agonist is alpha FP20.

Preferred embodiments include methods wherein said toll like receptor 4 agonist is FP200.

Preferred embodiments include methods wherein said therapeutic cell is a pluripotent interleukin-1 receptor antagonist upon stimulation with lipopolysaccharide.

Preferred embodiments include methods wherein said therapeutic cell is a pluripotent stem cell derived mesenchymal stem cell wherein said mesenchymal stem cell expresses interleukin-1 receptor antagonist upon stimulation with beta glucan.

Preferred embodiments include methods wherein said therapeutic cell is a pluripotent stem cell derived mesenchymal stem cell wherein said mesenchymal stem cell expresses interleukin-1 receptor antagonist upon stimulation with interferon gamma.

Preferred embodiments include methods wherein said therapeutic cell is a pluripotent stem cell derived mesenchymal stem cell wherein said mesenchymal stem cell expresses interleukin-1 receptor antagonist upon stimulation with TNF-alpha.

Preferred embodiments include methods wherein said therapeutic cell is a pluripotent stem cell derived mesenchymal stem cell wherein said mesenchymal stem cell expresses interleukin-1 receptor antagonist upon stimulation with lymphotoxin.

Preferred embodiments include methods wherein said therapeutic cell is a pluripotent stem cell derived mesenchymal stem cell wherein said mesenchymal stem cell expresses interleukin-1 receptor antagonist upon stimulation with TRANCE.

Preferred embodiments include methods wherein said therapeutic cell is a pluripotent stem cell derived mesenchymal stem cell wherein said mesenchymal stem cell expresses interleukin-1 receptor antagonist upon stimulation with free histone DNA.

Preferred embodiments include methods wherein said therapeutic cell is a pluripotent stem cell derived mesenchymal stem cell wherein said mesenchymal stem cell expresses interleukin-1 receptor antagonist upon stimulation with free DNA.

Preferred embodiments include methods wherein said therapeutic cell is a pluripotent stem cell derived mesenchymal stem cell wherein said mesenchymal stem cell expresses interleukin-1 receptor antagonist upon stimulation with double stranded RNA.

Preferred embodiments include methods wherein said therapeutic cell is a pluripotent interleukin-1 receptor antagonist upon stimulation with Poly IC.

Preferred embodiments include methods wherein said therapeutic cell is a pluripotent interleukin-1 receptor antagonist upon stimulation with Poly LC.

Preferred embodiments include methods wherein said therapeutic cell is a pluripotent stem cell derived mesenchymal stem cell wherein said mesenchymal stem cell expresses interleukin-1 receptor antagonist upon stimulation with CpG DNA.

Preferred embodiments include methods wherein said therapeutic cell is a pluripotent stem cell derived mesenchymal stem cell wherein said mesenchymal stem cell expresses interleukin-1 receptor antagonist upon stimulation with HMGB1.

Preferred embodiments include methods wherein said therapeutic cell is a pluripotent stem cell derived mesenchymal stem cell wherein said mesenchymal stem cell expresses VEGF-A upon stimulation with lipopolysaccharide.

Preferred embodiments include methods wherein said therapeutic cell is a pluripotent stem cell derived mesenchymal stem cell wherein said mesenchymal stem cell expresses VEGF-A upon stimulation with beta glucan.

Preferred embodiments include methods wherein said therapeutic cell is a pluripotent stem cell derived mesenchymal stem cell wherein said mesenchymal stem cell expresses VEGF-A upon stimulation with interferon gamma.

Preferred embodiments include methods wherein said therapeutic cell is a pluripotent stem cell derived mesenchymal stem cell wherein said mesenchymal stem cell expresses VEGF-A upon stimulation with TNF-alpha.

Preferred embodiments include methods wherein said therapeutic cell is a pluripotent stem cell derived mesenchymal stem cell wherein said mesenchymal stem cell expresses VEGF-A upon stimulation with lymphotoxin.

Preferred embodiments include methods wherein said therapeutic cell is a pluripotent stem cell derived mesenchymal stem cell wherein said mesenchymal stem cell expresses VEGF-A upon stimulation with TRANCE.

Preferred embodiments include methods wherein said therapeutic cell is a pluripotent stem cell derived mesenchymal stem cell wherein said mesenchymal stem cell expresses VEGF-A upon stimulation with free histone DNA.

Preferred embodiments include methods wherein said therapeutic cell is a pluripotent stem cell derived mesenchymal stem cell wherein said mesenchymal stem cell expresses VEGF-A upon stimulation with free DNA.

Preferred embodiments include methods wherein said therapeutic cell is a pluripotent stem cell derived mesenchymal stem cell wherein said mesenchymal stem cell expresses VEGF-A upon stimulation with double stranded RNA.

Preferred embodiments include methods wherein said therapeutic cell is a pluripotent stem cell derived mesenchymal stem cell wherein said mesenchymal stem cell expresses VEGF-A upon stimulation with Poly IC.

Preferred embodiments include methods wherein said therapeutic cell is a pluripotent stem cell derived mesenchymal stem cell wherein said mesenchymal stem cell expresses VEGF-A upon stimulation with Poly LC.

Preferred embodiments include methods wherein said therapeutic cell is a pluripotent stem cell derived mesenchymal stem cell wherein said mesenchymal stem cell expresses VEGF-A upon stimulation with CpG DNA.

Preferred embodiments include methods wherein said therapeutic cell is a pluripotent stem cell derived mesenchymal stem cell wherein said mesenchymal stem cell expresses VEGF-A upon stimulation with HMGB1.

Preferred embodiments include methods wherein said therapeutic cell is a pluripotent stem cell derived mesenchymal stem cell wherein said mesenchymal stem cell expresses endoglin upon stimulation with lipopolysaccharide.

Preferred embodiments include methods wherein said therapeutic cell is a pluripotent stem cell derived mesenchymal stem cell wherein said mesenchymal stem cell expresses endoglin upon stimulation with beta glucan.

Preferred embodiments include methods wherein said therapeutic cell is a pluripotent stem cell derived mesenchymal stem cell wherein said mesenchymal stem cell expresses endoglin upon stimulation with interferon gamma.

Preferred embodiments include methods wherein said therapeutic cell is a pluripotent endoglin upon stimulation with TNF-alpha.

Preferred embodiments include methods wherein said therapeutic cell is a pluripotent endoglin upon stimulation with lymphotoxin.

Preferred embodiments include methods wherein said therapeutic cell is a pluripotent stem cell derived mesenchymal stem cell wherein said mesenchymal stem cell expresses endoglin upon stimulation with TRANCE.

Preferred embodiments include methods wherein said therapeutic cell is a pluripotent stem cell derived mesenchymal stem cell wherein said mesenchymal stem cell expresses endoglin upon stimulation with free histone DNA.

Preferred embodiments include methods wherein said therapeutic cell is a pluripotent stem cell derived mesenchymal stem cell wherein said mesenchymal stem cell expresses endoglin upon stimulation with free DNA.

Preferred embodiments include methods wherein said therapeutic cell is a pluripotent stem cell derived mesenchymal stem cell wherein said mesenchymal stem cell expresses endoglin upon stimulation with double stranded RNA.

Preferred embodiments include methods wherein said therapeutic cell is a pluripotent stem cell derived mesenchymal stem cell wherein said mesenchymal stem cell expresses endoglin upon stimulation with Poly IC.

Preferred embodiments include methods wherein said therapeutic cell is a pluripotent stem cell derived mesenchymal stem cell wherein said mesenchymal stem cell expresses endoglin upon stimulation with Poly LC.

Preferred embodiments include methods wherein said therapeutic cell is a pluripotent stem cell derived mesenchymal stem cell wherein said mesenchymal stem cell expresses endoglin upon stimulation with CpG DNA.

Preferred embodiments include methods wherein said therapeutic cell is a pluripotent stem cell derived mesenchymal stem cell wherein said mesenchymal stem cell expresses endoglin upon stimulation with HMGB1.

Preferred embodiments include methods wherein said therapeutic cell is a pluripotent angiopoietin upon stimulation with lipopolysaccharide.

Preferred embodiments include methods wherein said therapeutic cell is a pluripotent angiopoietin upon stimulation with beta glucan.

Preferred embodiments include methods wherein said therapeutic cell is a pluripotent stem cell derived mesenchymal stem cell wherein said mesenchymal stem cell expresses angiopoietin upon stimulation with interferon gamma.

Preferred embodiments include methods wherein said therapeutic cell is a pluripotent stem cell derived mesenchymal stem cell wherein said mesenchymal stem cell expresses angiopoietin upon stimulation with TNF-alpha.

Preferred embodiments include methods wherein said therapeutic cell is a pluripotent stem cell derived mesenchymal stem cell wherein said mesenchymal stem cell expresses angiopoietin upon stimulation with lymphotoxin.

Preferred embodiments include methods wherein said therapeutic cell is a pluripotent stem cell derived mesenchymal stem cell wherein said mesenchymal stem cell expresses angiopoietin upon stimulation with TRANCE.

Preferred embodiments include methods wherein said therapeutic cell is a pluripotent stem cell derived mesenchymal stem cell wherein said mesenchymal stem cell expresses angiopoietin upon stimulation with free histone DNA.

Preferred embodiments include methods wherein said therapeutic cell is a pluripotent stem cell derived mesenchymal stem cell wherein said mesenchymal stem cell expresses angiopoietin upon stimulation with free DNA.

Preferred embodiments include methods wherein said therapeutic cell is a pluripotent stem cell derived mesenchymal stem cell wherein said mesenchymal stem cell expresses angiopoietin upon stimulation with double stranded RNA.

Preferred embodiments include methods wherein said therapeutic cell is a pluripotent stem cell derived mesenchymal stem cell wherein said mesenchymal stem cell expresses angiopoietin upon stimulation with Poly IC.

Preferred embodiments include methods wherein said therapeutic cell is a pluripotent angiopoietin upon stimulation with Poly LC.

Preferred embodiments include methods wherein said therapeutic cell is a pluripotent angiopoietin upon stimulation with CpG DNA.

Preferred embodiments include methods wherein said therapeutic cell is a pluripotent stem cell derived mesenchymal stem cell wherein said mesenchymal stem cell expresses angiopoietin upon stimulation with HMGB1.

Preferred embodiments include methods wherein said mesenchymal stem cell is plastic adherent.

Preferred embodiments include methods wherein said mesenchymal stem cell is derived from an embryonic body.

Preferred embodiments include methods wherein said embryoid body is derived from a pluripotent stem cell.

Preferred embodiments include methods wherein said pluripotent stem cell is a cell population possessing ability to proliferate indefinitely without senescence.

Preferred embodiments include methods wherein said pluripotent stem cell is a cell population possessing ability to proliferate indefinitely without telomere shortening.

Preferred embodiments include methods wherein said pluripotent stem cell is a cell population possessing ability to proliferate indefinitely without loss of differentiation ability.

Preferred embodiments include methods wherein said pluripotent stem cell is a cell population possessing ability to induce formation of a teratoma when administered to immune deficient animals.

Preferred embodiments include methods wherein said teratoma contains mesoderm, ectoderm and endoderm tissue.

Preferred embodiments include methods wherein said teratoma expresses the protein Fas ligand on its surface.

Preferred embodiments include methods wherein said teratoma expresses the protein MMP3 on its surface.

Preferred embodiments include methods wherein said teratoma expresses the protein MMP4 on its surface.

Preferred embodiments include methods wherein said teratoma expresses the protein MMP9 on its surface.

Preferred embodiments include methods wherein said teratoma expresses the protein c-met on its surface.

Preferred embodiments include methods wherein said teratoma expresses the protein CD47 on its surface.

Preferred embodiments include methods wherein said pluripotent stem cell expresses at least two of the following markers: a) OCT4; b) SOX2; c) NANOG; d) k-RAS; e) PIM-1 and f) KLF.

Preferred embodiments include methods wherein said pluripotent stem cell expresses SSEA4.

Preferred embodiments include methods wherein said pluripotent stem cell expresses TRA-1.

Preferred embodiments include methods wherein said pluripotent stem cell is an embryonic stem cell.

Preferred embodiments include methods wherein said pluripotent stem cell is a parthenogenic derived stem cell.

Preferred embodiments include methods wherein said pluripotent stem cell is a stress induced stem cell.

Preferred embodiments include methods wherein said pluripotent stem cell is a somatic cell nuclear transfer derived cell.

Preferred embodiments include methods wherein said pluripotent stem cell is an induced pluripotent stem cell.

Preferred embodiments include methods wherein said pluripotent stem cell is a cytoplasm transfer derived stem cell.

Preferred embodiments include methods wherein said pluripotent stem cell is maintained in an immature state by culture in serum free and feeder free media.

Preferred embodiments include methods wherein said pluripotent stem cell is maintained on feeder cells.

Preferred embodiments include methods wherein said feeder cells are allogeneic to the stem cell.

Preferred embodiments include methods wherein said feeder cells are xenogeneic to the stem cell.

Preferred embodiments include methods wherein said feeder cells are fibroblasts.

Preferred embodiments include methods wherein said feeder cells are mesenchymal stem cells.

Preferred embodiments include methods wherein said mesenchymal stem cells are bone marrow derived.

Preferred embodiments include methods wherein said bone marrow derived mesenchymal stem cells are selected for by expression of CD69.

Preferred embodiments include methods wherein said bone marrow derived mesenchymal stem cells are selected for by expression of CD47.

Preferred embodiments include methods wherein said bone marrow derived mesenchymal stem cells are selected for by expression of DAF1.

Preferred embodiments include methods wherein said bone marrow derived mesenchymal stem cells are selected for by expression of trkA (NGF receptor).

Preferred embodiments include methods wherein said bone marrow derived mesenchymal stem cells are selected for by expression of CD37.

Preferred embodiments include methods wherein said bone marrow derived mesenchymal stem cells are selected for by expression of c-kit.

Preferred embodiments include methods wherein said mesenchymal stem cells are umbilical cord tissue derived.

Preferred embodiments include methods wherein said umbilical cord tissue stem cells are selected for by expression of CD69.

Preferred embodiments include methods wherein said umbilical cord tissue stem cells are selected for by expression of CD47.

Preferred embodiments include methods wherein said umbilical cord tissue stem cells are selected for by expression of DAF1.

Preferred embodiments include methods wherein said umbilical cord tissue stem cells are selected for by expression of trkA (NGF receptor).

Preferred embodiments include methods wherein said umbilical cord tissue stem cells are selected for by expression of CD37.

Preferred embodiments include methods wherein said umbilical cord tissue stem cells are selected for by expression of c-kit.

Preferred embodiments include methods wherein said mesenchymal stem cells are adipose tissue derived.

Preferred embodiments include methods wherein said adipose tissue stem cells are selected for by expression of CD69.

Preferred embodiments include methods wherein said adipose tissue stem cells are selected for by expression of CD47.

Preferred embodiments include methods wherein said adipose tissue stem cells are selected for by expression of DAF1.

Preferred embodiments include methods wherein said adipose tissue stem cells are selected for by expression of trkA (NGF receptor).

Preferred embodiments include methods wherein said adipose tissue stem cells are selected for by expression of CD37.

Preferred embodiments include methods wherein said adipose tissue stem cells are selected for by expression of c-kit.

Preferred embodiments include methods wherein said therapeutic cell is a mesenchymal stem cell engineered to express a chimeric antigen receptor.

Preferred embodiments include methods wherein said chimeric antigen receptor comprises of an immunoglobulin domain and a T cell receptor signaling domain.

Preferred embodiments include methods wherein said chimeric antigen receptor binds to perispinal associated antigens.

Preferred embodiments include methods wherein said perispinal associated antigen is found on the annulus pulposus.

Preferred embodiments include methods wherein said perispinal associated antigen is aggrecan.

Preferred embodiments include methods wherein said perispinal associated antigen is hyaluronic acid.

Preferred embodiments include methods wherein said perispinal associated antigen is hyaluronic acid degradation fragments.

Preferred embodiments include methods wherein said hyaluronic acid degradation fragments are generated by hyaluronidase cleavage associated with perispinal inflammation.

Preferred embodiments include methods wherein said perispinal inflammation is enhanced local concentration of TNF-alpha as compared to age-matched health perispinal tissue.

Preferred embodiments include methods wherein said perispinal inflammation is enhanced local concentration of lymphotoxin as compared to age-matched health perispinal tissue.

Preferred embodiments include methods wherein said perispinal inflammation is enhanced local concentration of alpha 1 antitrypsin as compared to age-matched health perispinal tissue.

Preferred embodiments include methods wherein said perispinal inflammation is enhanced local concentration of MMP3 as compared to age-matched health perispinal tissue.

Preferred embodiments include methods wherein said perispinal inflammation is enhanced local concentration of MMP5 as compared to age-matched health perispinal tissue.

Preferred embodiments include methods wherein said perispinal inflammation is enhanced local concentration of MMP7 as compared to age-matched health perispinal tissue.

Preferred embodiments include methods wherein said perispinal inflammation is enhanced local concentration of MMP9 as compared to age-matched health perispinal tissue.

Preferred embodiments include methods wherein said perispinal inflammation is enhanced local concentration of interleukin 1 beta as compared to age-matched health perispinal tissue.

Preferred embodiments include methods wherein said perispinal inflammation is enhanced local concentration of neutrophil elastase as compared to age-matched health perispinal tissue.

Preferred embodiments include methods wherein said perispinal inflammation is enhanced local concentration of stomalysin as compared to age-matched health perispinal tissue.

Preferred embodiments include methods wherein said cell engineered to express said CAR is also modified to possess enhanced proclivity towards hypoxia.

Preferred embodiments include methods wherein said proclivity towards hypoxia is mediated by augmented expression of receptors mediating chemotaxis towards messengers released by hypoxic cells.

Preferred embodiments include methods wherein said messenger released by said hypoxic cells is VEGF-A.

Preferred embodiments include methods wherein said messenger released by said hypoxic cells is VEGF-C.

Preferred embodiments include methods wherein said messenger released by said hypoxic cells is kisspeptin.

Preferred embodiments include methods wherein said messenger released by said hypoxic cells is vasoactive intestinal peptide.

Preferred embodiments include methods wherein said messenger released by said hypoxic cells is acidic fibroblast growth factor.

Preferred embodiments include methods wherein said messenger released by said hypoxic cells is basic fibroblast growth factor.

Preferred embodiments include methods wherein said messenger released by said hypoxic cells is FGF-5.

Preferred embodiments include methods wherein said messenger released by said hypoxic cells is stromal derived factor 1.

Preferred embodiments include methods wherein said messenger released by said hypoxic cells is insulin like growth factor.

Preferred embodiments include methods wherein said messenger released by said hypoxic cells is insulin like growth factor binding protein.

Preferred embodiments include methods wherein said messenger released by said hypoxic cells is interleukin-8.

Preferred embodiments include methods wherein said messenger released by said hypoxic cells is macrophage inflammatory protein 1 alpha.

Preferred embodiments include methods wherein said messenger released by said hypoxic cells is macrophage inflammatory protein 1 beta.

Preferred embodiments include methods wherein said messenger released by said hypoxic cells is macrophage inflammatory protein 2.

Preferred embodiments include methods wherein said messenger released by said hypoxic cells is CCR7 ligand.

Preferred embodiments include methods wherein said messenger released by said hypoxic cells is kynurenine.

Preferred embodiments include methods wherein said messenger released by said hypoxic cells is quinolinic acid.

Preferred embodiments include methods wherein said messenger released by said hypoxic cells is galectin-1.

Preferred embodiments include methods wherein said messenger released by said hypoxic cells is galectin-3.

Preferred embodiments include methods wherein said messenger released by said hypoxic cells is galectin-7.

Preferred embodiments include methods wherein said retention of said therapeutic cell is enhanced by local administration in the perispinal area a chemokine producing means to enhance migration to, and retention of therapeutic cells to said perispinal area.

Preferred embodiments include methods wherein wherein said chemokine producing means is a cellular population.

Preferred embodiments include methods wherein said cellular population is a population of cells containing predominantly monocytes.

Preferred embodiments include methods wherein wherein said monocytes are plastic adherent.

Preferred embodiments include methods wherein said monocytes express CD16.

Preferred embodiments include methods wherein said monocytes express CD14.

Preferred embodiments include methods wherein said monocytes express CD163.

Preferred embodiments include methods wherein said monocytes express DEC205.

Preferred embodiments include methods wherein said monocytes express arginase.

Preferred embodiments include methods wherein said monocytes express CD40.

Preferred embodiments include methods wherein said monocytes express inducible nitric oxide synthase.

Preferred embodiments include methods wherein said monocytes express LRP.

Preferred embodiments include methods wherein said monocytes express TNF receptor p55.

Preferred embodiments include methods wherein said monocytes express TNF receptor p75.

Preferred embodiments include methods wherein said monocytes are generated from bone marrow progenitor cells.

Preferred embodiments include methods wherein said bone marrow progenitor cells express CD34.

Preferred embodiments include methods wherein said bone marrow progenitor cells express c-kit.

Preferred embodiments include methods wherein said bone marrow progenitor cells express CD90.

Preferred embodiments include methods wherein said bone marrow progenitor cells express IL-3 receptor.

Preferred embodiments include methods wherein said bone marrow progenitor cells express aldehyde dehydrogenase.

Preferred embodiments include methods wherein said bone marrow progenitor cells express CD133.

Preferred embodiments include methods wherein said bone marrow progenitor cells are differentiated into monocytes by culture in M-CSF for a sufficient time period and concentration to induce differentiation of at least 5% of said bone marrow hematopoietic progenitor cells into cells expressing CD14.

Preferred embodiments include methods wherein said bone marrow progenitor cells are differentiated into monocytes by culture in M-CSF for a sufficient time period and concentration to induce differentiation of at least 50% of said bone marrow hematopoietic progenitor cells into cells expressing CD14.

Preferred embodiments include methods wherein said bone marrow progenitor cells are differentiated into monocytes by culture in M-CSF for a sufficient time period and concentration to induce differentiation of at least 50% of said bone marrow hematopoietic progenitor cells into cells expressing CD14.

Preferred embodiments include methods wherein said bone marrow progenitor cells are also cultured in IL-3.

Preferred embodiments include methods wherein said bone marrow progenitor cells are also cultured in IL-10.

Preferred embodiments include methods wherein said bone marrow progenitor cells are also cultured in IL-3 and TNF-alpha.

Preferred embodiments include methods wherein said bone marrow progenitor cells are also cultured in IL-3.

Preferred embodiments include methods wherein said monocyte is transfected with one or more genes capable of stimulating mesenchymal stem cell chemoattraction and retention.

Preferred embodiments include methods wherein said monocyte is transfected with one or more genes capable of stimulating T regulatory cell chemoattraction and retention.

Preferred embodiments include methods wherein said mesenchymal stem cell chemoattractant is CXCL12.

Preferred embodiments include methods wherein said mesenchymal stem cell chemoattractant is TRANCE.

Preferred embodiments include methods wherein said mesenchymal stem cell chemoattractant is DC-SIGN.

Preferred embodiments include methods wherein said mesenchymal stem cell chemoattractant is TRAIL.

Preferred embodiments include methods wherein said mesenchymal stem cell chemoattractant is nitric oxide.

Preferred embodiments include methods wherein said mesenchymal stem cell chemoattractant is oxidated LDL.

Preferred embodiments include methods wherein said mesenchymal stem cell chemoattractant is RANK ligand.

Preferred embodiments include methods wherein said mesenchymal stem cell chemoattractant is HMGB1.

Preferred embodiments include methods wherein said mesenchymal stem cell chemoattractant is prostacyclin.

Preferred embodiments include methods wherein said mesenchymal stem cell chemoattractant is PGE1

Preferred embodiments include methods wherein said mesenchymal stem cell chemoattractant is PGE2.

Preferred embodiments include methods wherein said mesenchymal stem cell chemoattractant is uric acid.

Preferred embodiments include methods wherein said mesenchymal stem cell chemoattractant is chemerin.

Preferred embodiments include methods wherein said mesenchymal stem cell chemoattractant is CCL27.

Preferred embodiments include methods wherein said mesenchymal stem cell chemoattractant is CCL21.

Preferred embodiments include methods wherein said gene capable of stimulating T regulatory cell chemoattraction and retention is CCL7.

Preferred embodiments include methods wherein said gene capable of stimulating T regulatory cell chemoattraction and retention is interleukin-2.

Preferred embodiments include methods wherein said gene capable of stimulating T regulatory cell chemoattraction and retention is interleukin-6.

Preferred embodiments include methods wherein said gene capable of stimulating T regulatory cell chemoattraction and retention is interleukin-7.

Preferred embodiments include methods wherein said gene capable of stimulating T regulatory cell chemoattraction and retention is interleukin-10.

Preferred embodiments include methods wherein said gene capable of stimulating T regulatory cell chemoattraction and retention is interleukin-13.

Preferred embodiments include methods wherein said gene capable of stimulating T regulatory cell chemoattraction and retention is interleukin-20.

Preferred embodiments include methods wherein said gene capable of stimulating T regulatory cell chemoattraction and retention is interleukin-22.

Preferred embodiments include methods wherein said gene capable of stimulating T regulatory cell chemoattraction and retention is interleukin-35.

Preferred embodiments include methods wherein said gene capable of stimulating T regulatory cell chemoattraction and retention is leukemia inhibitory factor.

Preferred embodiments include methods wherein said gene capable of stimulating T regulatory cell chemoattraction and retention is TGF-beta.

Preferred embodiments include methods wherein said gene capable of stimulating T regulatory cell chemoattraction and retention is OX2.

Preferred embodiments include methods wherein said gene capable of stimulating T regulatory cell chemoattraction and retention is Tim-3.

Preferred embodiments include methods wherein said gene capable of stimulating T regulatory cell chemoattraction and retention is soluble HLA-G.

Preferred embodiments include methods wherein said gene capable of stimulating T regulatory cell chemoattraction and retention is endoglin.

Preferred embodiments include methods wherein said gene capable of stimulating T regulatory cell chemoattraction and retention is HIF-alpha.

Preferred embodiments include methods wherein said gene capable of stimulating T regulatory cell chemoattraction and retention is NF-kappa B.

Preferred embodiments include methods wherein said gene capable of stimulating T regulatory cell chemoattraction and retentin is RelB.

Preferred embodiments include methods wherein said gene capable of stimulating T regulatory cell chemoattraction and retention is AIRE.

Preferred embodiments include methods wherein said gene capable of stimulating T regulatory cell chemoattraction and retention is FoxP3.

Preferred embodiments include methods wherein said gene capable of stimulating T regulatory cell chemoattraction and retention is heme oxygenase-1.

Preferred embodiments include methods wherein said cellular population is a population of cells containing predominantly T cells.

Preferred embodiments include methods wherein said T cell is derived from the thymus.

Preferred embodiments include methods wherein said T cell is derived from peripheral circulation.

Preferred embodiments include methods wherein said T cell is derived from the spleen.

Preferred embodiments include methods wherein said T cell is derived from the lymph nodes.

Preferred embodiments include methods wherein said T cell is derived from adipose tissue.

Preferred embodiments include methods wherein said T cell is derived from pluripotent stem cells.

Preferred embodiments include methods wherein said T cell is a CD3 expressing cell.

Preferred embodiments include methods wherein said T cell is a CD4 expressing cell.

Preferred embodiments include methods wherein said CD4 T cell is a Th1 cell.

Preferred embodiments include methods wherein said Th1 cells possesse an increased

proclivity towards producing interferon gamma as compared to interleukin-4.

Preferred embodiments include methods wherein said Th1 cells possess an increased proclivity towards producing interferon gamma as compared to interleukin-10.

Preferred embodiments include methods wherein said Th1 cells possess an increased proclivity towards producing interferon gamma as compared to interleukin-13.

Preferred embodiments include methods wherein said Th1 cells possess an increased proclivity towards producing interferon gamma as compared to interleukin-20.

Preferred embodiments include methods wherein said Th1 cells possess an increased proclivity towards producing interferon gamma as compared to interleukin-35.

Preferred embodiments include methods wherein said Th1 cells possess an increased proclivity towards inducing activation of dendritic cell maturation.

Preferred embodiments include methods wherein said dendritic cell maturation is associated with augmented antigen presenting activity.

Preferred embodiments include methods wherein said dendritic cell maturation is associated with diminished migratory activity.

Preferred embodiments include methods wherein said dendritic cell maturation is associated with augmented expression of CD40.

Preferred embodiments include methods wherein said dendritic cell maturation is associated with augmented expression of CD80.

Preferred embodiments include methods wherein said dendritic cell maturation is associated with augmented expression of CD86.

Preferred embodiments include methods wherein said dendritic cell maturation is associated with augmented expression of CD40.

Preferred embodiments include methods wherein said Th1 cells possess an increased proclivity towards stimulating cytotoxicity of CD8 T cells.

Preferred embodiments include methods wherein said cytotoxicity is associated with enhanced production of granzyme B.

Preferred embodiments include methods wherein said cytotoxicity is associated with enhanced production of perforin.

Preferred embodiments include methods wherein said degenerative disc disease is associated with reduced oxygen tension in the annulus fibrosis.

Preferred embodiments include methods wherein said degenerative disc disease is associated with reduced oxygen tension in the nucleus pulposus.

Preferred embodiments include methods wherein said degenerative disc disease is associated with occlusion of the perispinal arteries.

Preferred embodiments include methods wherein said mesenchymal stem cell population is generated from a pluripotent stem cell source.

Preferred embodiments include methods wherein said pluripotent stem cell source is embryonic stem cells.

Preferred embodiments include methods wherein said pluripotent stem cell source is parthenogenic derived stem cells.

Preferred embodiments include methods wherein said pluripotent stem cell source is somatic cell nuclear transfer derived stem cells.

Preferred embodiments include methods wherein said pluripotent stem cell source is stem cells generated by introduction of cytoplasm from an undifferentiated cell to a differentiated cell.

Preferred embodiments include methods wherein said pluripotent stem cell is an induced pluripotent stem cell.

Preferred embodiments include methods wherein said induced pluripotent stem cells are generated by transfection of a somatic cell with one or more dedifferentiation factors.

Preferred embodiments include methods wherein said dedifferentiation factor is a microRNA.

Preferred embodiments include methods wherein said dedifferentiation factor is OCT4.

Preferred embodiments include methods wherein said dedifferentiation factor is NANOG.

Preferred embodiments include methods wherein said dedifferentiation factor is KLF.

Preferred embodiments include methods wherein said dedifferentiation factor is Sox-2.

Preferred embodiments include methods wherein said dedifferentiation factor is Ras.

Preferred embodiments include methods wherein said somatic cell is selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred embodiments include methods wherein said somatic cell is a progenitor cell which lacks pluripotency.

Preferred embodiments include methods wherein said somatic cell to be programmed into induced pluripotent stem cell is an endothelial progenitor cell.

### DETAILED DESCRIPTION OF THE INVENTION

The invention provides means of stimulating the immune system in a tolerogenic/anti-inflammatory manner to allow reduction of pathology and provide stimulatory means for initiation of angiogenesis and regenerative mechanisms. The invention provides the specific administration of mesenchymal stem cell and T regulatory cells for treatment of disc degeneration. Specifically, the invention provides means of treating and/or reversing disc degeneration by utilizing pluripotent stem cells as originating cells for the generation of mesenchymal stem cells and/or T regulatory cells. Furthermore, the invention provides mesenchymal stem cells, or MSCs, including human mesenchymal stem cells that have been generated from pluripotent stem cells or other progenitors. More particularly, this invention relates to mesenchymal stem cells produced from induced pluripotent stem cells (iPSCs), and in some embodiments in which said cells are generated to possess one or more chimeric antigen receptors (CAR). In some embodiments therapeutic effects of said mesenchymal stem cells or CAR-mesenchymal stem cells are enhanced by coadministration of T regulatory cells. Said T regulatory cells may be generated from existing peripheral blood or other sources, as well as pluripotent induced.

In some embodiments iPSCs are cultured under conditions to provide mesenchymal stem cells that are less likely to produce tumors or cancers and are more stable as compared to administration of iPSC. This is particularly important in conditions of chronic lower back pain which is associated with low level inflammation which possesses possibility of triggering dormant tumors.

The iPSCs can be cultured using suitable culturing conditions. For example, iPSCs can be maintained using protocols such as those disclosed in Gao Y, Guo X, Santostefano K et al. Genome Therapy of Myotonic Dystrophy Type 1 iPS Cells for Development of Autologous Stem Cell Therapy. Mol Ther. 2016; 24:1378-1387; Xia G, Gao Y, Jin S et al. Genome modification leads to phenotype reversal in human myotonic dystrophy type 1 induced pluripotent stem cell-derived neural stem cells. Stem Cells. 2015; 33:1829-1838; Xia G, Santostefano K, Hamazaki T et al. Generation of human-induced pluripotent stem cells to model spinocerebellar ataxia type 2 in vitro. J Mol Neurosci. 2013; 51:237-248; and Xia G, Santostefano K E, Goodwin M et al. Generation of neural cells from DM1 induced pluripotent stem cells as cellular model for the study of central nervous system neuropathogenesis. Cell Reprogram. 2013; 15:166-177, each of which is incorporated by reference. According to some embodiments, these protocols may be modified to meet the criteria of clinically-clean iPSCs, including the use of feeder-free, xeno-free culture and coating media. While common cultures call for the use of an extracellular matrix such as, for example, the Corning Matrigel matrix (Corning, New York, N.Y.), it should be noted that the Corning Matrigel matrix contains a mixture of matrix proteins and growth factors of non-human origin. Accordingly, for applications wherein the cells are ultimately to be implanted in a human subject, it may be desirable to use cultures conditions that do not utilize non-human origin additives. According to a specific example, cultured cells may be coated with laminin and collagen IV from human cell culture (for example, Sigma-Aldrich C6745, Sigma-Aldrich Co.) and adapted to Laminin 521 coating culture conditions. Laminin 521 (LaminStem™ 521,05-753-1F, Biological Industries) is a chemically defined, animal component-free, xeno-free matrix. Those of skill in the art will be familiar with other suitable culturing conditions as well as the adaptation of those conditions for the specific uses of the presently described genome corrected cells.

Prior to being cultured under conditions to produce mesenchymal stem cells, the iPSCs may be genetically engineered with at least one polynucleotide encoding at least one biologically active protein or polypeptide or biologically active fragment, derivative, or analogue thereof, thus enabling one to produce genetically engineered mesenchymal stem cells from the genetically engineered iPSCs that express sustained levels of the at least one biologically active protein or polypeptide, or biologically active fragment, derivative, or analogue thereof. This allows for the generation of iPSCs which can be selectively targeting to the area of inflammation. For example, in embodiment iPSCs are engineered to selectively engage antigens associated with the perispinal area of the degenerating disc.

Moreover, MSCs have a limited proliferation potential as they are expanded. This provides an added level of protection from neoplastic transformation.

For the treatment of lower back pain the invention teaches the stimulation of angiogenesis. This is accomplished in the invention by transfecting MSC with CAR, as well as enhancing receptor expression, for example expression of CXCR4. In order to address the limitations of expandability and standardization, MSCs are derived, for the purpose of the invention, from induced pluripotent stem cells (iPSCs) with a modified protocol that can be expanded to provide large cell banks from a single cell clone. The protocol produces highly enriched MSC-like cells from iPSCs with high efficiency. The iPSC-derived MSCs (iPSC-MSCs) express the classical surface markers of MSCs, are capable of multi-lineage mesodermal differentiation and cancer homing, can be expanded extensively, but do not preserve the pluripotency of iPSCs. The data indicated that iPSC-MSCs are a safe alternative to BM-MSCs for patients at potential risk of cancer. In accordance with an aspect of the present invention, there is provided a method of producing mesenchymal stem cells from induced pluripotent stem cells. The method comprises culturing the induced pluripotent stem cells in a medium containing a TGF-6 inhibitor (also known as an Smad 2/3 pathway) inhibitor and in an atmosphere containing from about 7 vol % to about 8 vol. % carbon dioxide (CO2) for a period of time of from about 20 days to about 35 days. The cells then are transfered to a culture dish having a hydrophilic surface, and the cells are cultured in a medium containing a TGF-β inhibitor for a period of time sufficient to produce mesenchymal stem cells. The mesenchymal stems cells then may be isolated from the culture medium by means known to those skilled in the art. In a non-limiting embodiment, the mesenchymal stem cells are mammalian mesenchymal stem cells produced from mammalian induced pluripotent stem cells. In another non-limiting embodiment, the mammal is a primate. In yet another non-limiting embodiment, the primate is a human. The TGF-β inhibitor may, in a non-limiting embodiment, be selected from those known to those skilled in the art. In a non-limiting embodiment, the TGF-β inhibitor is a product known as SB-431542 sold by Sigma-Aldrich, St. Louis, Mo.

In another non-limiting embodiment, the induced pluripotent stem cells are cultured in an atmosphere containing about 7.5 vol. % CO2. In another non-limiting embodiment, the induced pluripotent stem cells are cultured in the medium containing the TFG-β inhibitor and in the atmosphere containing from about 7wt. % to about 8 wt. % CO2 for a period of time of about 25 days.

In another non-limiting embodiment, after the cells are cultured in the medium containing the TGF-β inhibitor and in an atmosphere containing from about 7 vol. % to about 8 vol. % CO2 for from about 20 days to about 35 days, the cells are transferred to a culture dish having an oxygenated surface, which makes the surface hydrophilic. Such culture dishes in general may be standard tissue culture plastic dishes known to those skilled in the art. In such culture dishes, there is a culture medium containing a TGF-β inhibitor, such as SB-43152, for example. In another non-limiting embodiment, the cells are cultured in such culture dish and in the medium containing a TGF-β inhibitor for a period of time of about 21 days, thereby providing a culture of mesenchymal stem cells derived from induced pluripotent stem cells. In a non-limiting embodiment, induced pluripotent stem cells are cultured in a medium, such as the feeder-free medium mTeSR1 (STEMCELL Technologies) that has been supplemented with a TGF-β inhibitor such as SB431542 in an atmosphere containing 7.5 vol. % CO2 for 25 days. The cells then are transfered to a tissue culture plastic dish having a hydrophilic surface, and which contains a medium, such as a modified human ES-MSC medium containing knockout serum replacement, nonessential amino acids, antibiotic such as penicillin and streptomycin, glutamine, β-mercaptoethanol, and bFGF, which has been supplemented with a TGF-β inhibitor such as SB-431542. The medium is changed daily, and the cells are passaged at 80%- 90% confluence about every 3 days. The cells are cultured for a total of about 21 days to provide a majority of cells that are positive for MSC surface markers. Such mesenchymal stem cells also are known as iPSC-MSCs. The iPSC-MSCs then can be cultured in the presence of a standard medium, such as 20% fetal bovine serum (FBS) α-MEM medium, and then harvested for further experiments or for use in treating diseases or disorders, or for regenerating cells, tissues, or organs.

The mesenchymal stem cells formed from the induced pluripotent stem cells in accordance with the present invention thus have several desirable properties and characteristics that make the mesenchymal stem cells more stable, and whereby such mesenchymal stem cells are less likely to form or cause tumors, cancers, or teratomas, and thus are more desirable for use in therapy than other mesenchymal stem cells. Thus, in accordance with another aspect of the present invention, there are provided isolated human mesenchymal stem cells derived from human induced pluripotent stem cells that express no more than 1% of the levels of the Nanog, October 4, Ecad, and Foxa2 genes than the induced pluripotent stem cells from which the mesenchymal stem cells were derived.

In a non-limiting embodiment, the isolated human mesenchymal stem cells are at least 95% positive for the epitopes CD73, CD105, and CD166. In another non-limiting embodiment, the isolated human mesenchymal stem cells are at least 85% positive for the epitopes CD44 and CD90. In yet another non-limiting embodiment, the isolated human mesenchymal stem cells are no more than 5% positive for the epitopes HLA-DR, CD11b, CD24, CD34, and CD45. Furthermore, the isolated human mesenchymal stem cells of the present invention, in a non-limiting embodiment, contain the following levels of messenger RNAs (mRNAs) relative to a standardized preparation of MSC obtained from bone marrow (Sample No. 7075, available from the Institute for Regenerative Medicine, Texas A&M College of Medicine, Temple, Texas 76502): about 80% to about 120% of the mesodermal marker CD140A, about 550% to about 650% of the angiogenic gene VEGF, and less than 20% ±5% of the following genes known to promote the growth and metastasis of cancer cells: ILR1, mPGES1, IL-6, TGF-13R2, ID3, SDF1, HAS1, and HAS2.

The isolated CAR mesenchymal or untransfected MSC stem cells of the present invention may be administered in an amount effective to treat a variety of diseases and disorders, and to regenerate a variety of cells, tissues, and organs. The isolated human mesenchymal stem cells may be administered systematically such as by intramuscular, intravenous, intraperitoneal or intra-arterial administration or may be administered directly to an affected cell, tissue, or organ. The isolated human mesenchymal stem cells may be administered in conjunction with an acceptable pharmaceutical carrier adjuvant or excipient known to those skilled in the art. The exact dosage of mesenchymal stem cells to be administered is dependent upon a variety of factors, including but not limited to the age, weight, height, and sex of the patient, the disease or disorder being treated, and the extent and severity thereof, or the cells, tissue, or organ to be regenerated.

It is to be understood, however, that the scope of the present invention is not intended to be limited to the treatment of any particular disease, condition, or disorder, or to the regeneration of any particular cell, tissue, or organ. The isolated human mesenchymal stem cells of the present invention, prepared as hereinabove described, and having the properties hereinabove described, may be genetically engineered with at least one polynucleotide encoding at least one biologically active protein or polypeptide or biologically active fragment, derivative, or analogue thereof. Thus, in accordance with an aspect of the present invention, there is provided a method of producing genetically engineered mesenchymal stem cells from induced pluripotent stem cells. The method comprises introducing into the induced pluripotent stem cells at least one polynucleotide encoding at least one biologically active protein or polypeptide, or biologically active fragment, analogue, or derivative thereof to provide genetically engineered induced pluripotent stem cells. The genetically engineered pluripotent stem cells then are cultured as hereinabove described to produce genetically engineered mesenchymal stem cells, such as mammalian mesenchymal stem cells. In a non-limiting embodiment, the genetically engineered mammalian mesenchymal stem cells are primate mesenchymal stem cells, including human mesenchymal stem cells.

Thus, the genetically engineered induced pluripotent stem cells are cultured in a medium containing a TGF-β inhibitor and in an atmosphere containing from about 7 vol. % to about 8 vol. % CO2 (about 7.5 vol. % CO2 in another non-limiting embodiment) for a period of time of from about 20 days to about 35 days (about 25 days in another non-limiting embodiment). The genetically engineered cells then are transferred to a culture dish having a hydrophilic surface, such as those hereinabove described, and cultured in a medium containing a TGF-β inhibitor for a period of time (in a non-limiting embodiment, 21 days) sufficient to produce genetically engineered mesenchymal stem cells.

The at least one polynucleotide including at least one biologically active protein or polypeptide or biologically active fragment or derivative may be in the form of DNA (including but not limited to genomic DNA (gDNA) or cDNA, or RNA. The at least one polynucleotide encoding at least one biologically active protein or polypeptide or biologically active fragment, derivative, or analogue thereof may be contained in an appropriate expression vector, such as an adenoviral vector, adeno-associated virus vector, retroviral vector, or lentiviral vector that is introduced into the induced pluripotent stem cells, or may be contained in a transposon that is introduced into the cell, or the at least one polynucleotide may be introduced into the cell as naked DNA or RNA. Such introduction of the at least one polynucleotide may be introduced into the cell by any of a variety of means known to those skilled in the art, such as calcium phosphate precipitation, liposomes, gene guns, or by clustered regularly interspersed short palindromic repeats, or CRISPR, technology.

Biologically active proteins or polypeptides, or biologically active fragments, derivatives, or analogues thereof that may be introduced into the induced pluripotent stem cells, prior to the production of mesenchymal stem cells therefrom, include polynucleotides encoding various therapeutic agents including, but not limited to, anti-inflammatory or inflammation modulatory agents, such as TSG-6, anti-angiogenic agents, tumor necrosis factors, interleukins, growth factors, anti-clotting agents, bone morphogenic proteins (BMPs), such as BMP-2, hormones, such as insulin, anti-tumor agents, and negative selective markers. It is to be understood, however, that the scope of the present invention is not intended to be limited to any particular biologically active protein or polypeptide, or biologically active fragment, derivative, or analogue thereof. In a non-limiting embodiment the at least one biologically active protein or polypeptide or biologically active fragment, derivative, or analogue is tumor necrosis factor alpha stimulating gene 6 (TSG-6) protein or a biologically active fragment, derivative, or analogue thereof.

In one embodiment of the invention, pluripotent stem cell derived mesenchymal stem cells alone or in combination with T regulatory cells are administered to provide a mitogenic stimuli for intervertebral disk nucleus pulposus cell (or the cell population comprising said cell). These cells can be activated in vivo or can be isolated from the intervertebral disk nucleus pulposus of vertebrates characterized by being positive for at least one surface marker from among Tie2 and GD2. In one embodiment of the invention, intervertebral disk nucleus pulposus stem cells are activated by perispinal administration of pluripotent stem cell derived CAR-MSC and/or T regulatory cells. Activation of notochord cells by mesenchymal stem cells is accomplished, in one embodiment, by the growth factors released from the cells. These growth factors include any material or materials having a positive reaction on living tissues, such as promoting the growth of tissues. Exemplary growth factors include, but are not limited to, platelet-derived growth factor (PDGF), platelet-derived angiogenesis factor (PDAF), vascular endothelial growth factor (VEGF), platelet-derived epidermal growth factor (PDEGF), platelet factor 4 (PF-4), transforming growth factor beta. (TGF-B), acidic fibroblast growth factor (FGF-A), basic fibroblast growth factor (FGF-B), transforming growth factor A (TGF-A), insulin-like growth factors 1 and 2 (IGF-1 and IGF-2), B thromboglobulin-related proteins (BTG), thrombospondin (TSP), fibronectin, von Wallinbrand's factor (vWF), fibropeptide A, fibrinogen, albumin, plasminogen activator inhibitor 1 (PAI-1), osteonectin, regulated upon activation normal T cell expressed and presumably secreted (RANTES), gro-A, vitronectin, fibrin D-dimer, factor V, antithrombin III, immunoglobulin-G (lgG), immunoglobulin-M (IgM), immunoglobulin-A (IgA), a2-macroglobulin, angiogenin, Fg-D, elastase, keratinocyte growth factor (KGF), epidermal growth factor (EGF), fibroblast growth factor (FGF), tumor necrosis factor (TNF), fibroblast growth factor (FGF) and interleukin-1 (IL-1), Keratinocyte Growth Factor-2 (KGF-2), and combinations thereof. One of the important characteristics common to the above listed growth factors is that each substance is known or believed to have a positive reaction on living tissue, known as bioactivity, to enhance cell or tissue growth. These are cells that are at least Tie2-positive for the surface marker and possesses self-renewal ability as well as multipotency capable of differentiating into adipocytes, osteocytes, chondrocytes, or. Among such stem cells, those which are GD2-negative for the surface marker are in a dormant state, and those which are GD2-positive are in an activated state. Moreover, another embodiment of the invention is the activation of said intervertebral disk nucleus pulposus cell which is a progenitor cell that is Tie2-negative and GD2-positive for the surface marker, and capable of differentiating into adipocytes, osteocytes, chondrocytes, or neurons (referred to as the “intervertebral disk nucleus pulposus progenitor cell” in the present invention). The invention teaches the stimulation of nucleus pulposus cells by administration of monocytes. Regarding said intervertebral disk nucleus pulposus stem cell, the surface marker is additionally CD24-negative, CD44-positive/negative (in the case of stem cells) or positive (in the case of progenitor cells), CD271-positive, and Flt1-positive. Moreover, regarding said intervertebral disk nucleus pulposus progenitor cell, the surface marker is additionally CD24-negative or positive, CD44-positive, CD271-positive/negative or negative, and Flt1-positive/negative or negative. In one embodiment of the invention, the present invention provides a cultivation method for the intervertebral disk nucleus pulposus cell (or the cell population including said cells), the method characterized by comprising: isolating a cell positive for at least one surface marker from among Tie2 and GD2 from a nucleus pulposus cell population collected from the intervertebral disk nucleus pulposus of the vertebrate or a cell population obtained by cultivating the same. One embodiment of said cultivation method of intervertebral disk nucleus pulposus cells is a cultivation method of the intervertebral disk nucleus pulposus stem cell wherein the isolated cell is at least Tie2-positive for the surface marker, and another aspect is a cultivation method of the intervertebral disk nucleus pulposus progenitor cell wherein the isolated cell is Tie2-negative and GD2-positive for the surface marker. The cultivation method of said intervertebral disk nucleus pulposus stem cell may comprise cultivating said cells in the presence of an angiopoietin I (Ang-1). The Tie2 is a receptor of angiopoietin I.

In one embodiment, monocytes are administered together with pluripotent derived MSC to enhance perispinal homing and retention of said regenerative cells. Monocytes may be obtained from a screened donor(s). In this embodiment, a screened donor provides tissue for expansion of monocytes and creation of a master cell bank (MCB). After appropriate tests are conducted on the MCB, cells expanded from the master bank are used to create a working cell bank (WCB). The manufacturing process is similar to the autologous process, has the same applications and all final formulations are within the same concentration ranges. Somatic cells transfected with retroviral vectors that express OCT4, SOX2, KLF4 and cMYC to generate induced pluripotent stem cells (“iPSCs”) express the same pluripotency markers as control H9 ESCs. Reprogrammed cells possess a normal karyotype, differentiate into beating cardiomyocytes in vitro and differentiate into representatives of all three germ layers in vivo. A subpopulation of human dermal monocytes that express the pluripotency marker stage specific embryonic antigen 3 (SSEA3) demonstrates enhanced iPSC generation efficiency as described by Bryne, et al., PLOS One, 4(9):e7118 (2009). SSEA3-positive and SSEA3-negative populations were transduced with the same retroviral vectors, under identical experimental conditions, and seeded onto inactivated mouse embryonic fibroblasts (MEFs). After three weeks of culture under standard hESC conditions, plates were examined in a double-blind analysis by three independent hESC biologists for iPSC colony formation. Colonies with iPSC morphology were picked and expanded. All three biological replicates with the transduced SSEA3-negative cells formed many large background colonies (10-27 per replicate) but no iPSC colonies emerged; in contrast, all three biological replicates with the transduced SSEA3-positive cells resulted in the formation of iPSC colonies (4-5 per replicate) but very few large background colonies (0-1 per replicate). Further characterization of the cell lines derived from the iPSC-like colonies showed that they possessed hESC-like morphology, growing as flat colonies with large nucleo-cytoplasmic ratios, defined borders and prominent nucleoli. When five lines were further expanded and characterized, all demonstrated expression of key pluripotency markers expressed by hESCs, which included alkaline phosphatase, Nanog, SSEA3, SSEA4, TRA160 and TRA181. The SSEA3-selected iPSCs also demonstrated a normal male karyotype (46, XY), the ability to differentiate into functional beating cardiomyocytes in vitro and differentiate into representatives of all three germ layers in vivo. Since no iPSC colony formation or line derivation from the transduced SSEA3-negative cells was observed, this indicates that these cells possess significantly lower or even no reprogramming potential relative to the SSEA3-expressing cells. Additionally, a 10-fold enrichment of primary monocytes that strongly express SSEA3 results in a significantly greater efficiency (8-fold increase) of iPSC line derivation compared to the control derivation rate (p<0.05). The SSEA3-positive cells appeared indistinguishable, morphologically, from the SSEA3-negative monocytes; furthermore, expression of the SSEA3 antigen is not considered a marker of other cell types such as mesenchymal or epidermal adult stem cells.

In one embodiment, the present invention provides the administration of pluripotent stem cell derived mesenchymal stem cells as a means of stimulating a cell composition characterized by comprising said intervertebral disk nucleus pulposus cell (stem cell and/or progenitor cell). Regarding such a cell composition, for example, those for treatment or prevention of intervertebral disk disorders are preferable. In one embodiment, the present invention provides a treatment or prevention method of intervertebral disc disorders in vertebrates comprising transplanting said intervertebral disk nucleus pulposus cells (stem cells and/or progenitor cells) or said cell composition on the intervertebral disk. Moreover, the present invention provides a treatment or prevention method of intervertebral disk disorders invertebrates comprising administrating Ang-1 to the intervertebral disk nucleus pulposus stem cell in the intervertebral disks of the living body. Furthermore, said treatment or prevention method of intervertebral disk disorders may be applied in the same manner to humans. In one embodiment, the present invention provides a method of obtaining an indicator related to the state of the intervertebral disk, comprising measuring the proportion of the intervertebral disk nucleus pulposus stem cells in a nucleus pulposus cell population sampled from the intervertebral disk nucleus pulposus of a vertebrate.

Treatment of patients with lower back pain may be accomplished through another embodiment of the invention disclosed through the administration of mesenchymal stem cells and in some embodiments together with T reuglatoyr cells, either naturally derived or iPSC derived that have been genetically modified to upregulate expression of angiogenic stimuli or anti-inflammatory activities. It is known in the art that genes may be introduced by a wide range of approaches including adenoviral, adeno-associated, retroviral, alpha-viral, lentiviral, Kunjin virus, or HSV vectors, liposomal, nano-particle mediated as well as electroporation and Sleeping Beauty transposons. Genes with angiogenic stimulatory function that may be transfected include but are not limited to: VEGF, FGF-1, FGF-2, FGF-4, EGF, and HGF. Additionally, transcription factors that are associated with upregulating expression of angiogenic cascades may also be transfected into cells used for treatment of lower back pain, said genes include: HIF-1 [symbol] , HIF-2, NET, and NF-KB. Genes inhibitory to inflammation may be used such as: TGF-a, TGF-b, IL-4, IL-10, IL-13, IL-20 or thrombospondin. Transfection may also be utilized for administration of genetic manipulation means in a manner to substantially block transcription or translation of genes which inhibit angiogenesis. Antisense oligonucleotides, ribozymes or short interfering RNA may be transfected into cells for use for treatment of lower back pain in order to block expression of antiangiogenic proteins such as: canstatin, IP-10, kringle 1-5, and collagen XVIII/endostatin. Additionally, said gene inhibitory technologies may be used for blocking ability of cells to be used for treatment of lower back pain to express inflammatory proteins including: IL-1, TNF- [symbol] , IL-2, IL-6, IL-8, IL-9, IL-11, IL-12, IL-15, IL-17, IL-18, IL-21, IL-23, IL-27, IFN- [symbol] , IFN- [symbol] , and IFN- [symbol] . Globally acting transcription factors associated with inflammation may also be substantially blocked using not only the genetic means described but also decoy oligonucleotides. Suitable transcription factors for blocking include various subunits of the NF-kB complex such as p55, and/or p60, STAT family members, particularly STAT1, STAT5, STAT4, and members of the Interferon Regulatory Factor family such as IRF-1, IFR-3, and IFR-8. Enhancement of angiogenic stimulation ability of said cells useful for the treatment of back pain can be performed through culturing under conditions of restricted oxygen. It is known in the art that stem cells in general, and ones with angiogenesis promoting activity specifically, tend to reside in hypoxia niches of the bone marrow. When stem cells differentiate into more mature progeny, they progressively migrate to areas of the bone marrow with higher oxygen tension. This important variable in tissue culture was used in studies that demonstrated superior expansion of human CD34 stem cells capable of full hematopoietic reconstitution were obtained in hypoxic conditions using oxygen tension as low as 1.5%. The potent expansion under hypoxia, which was 5.8-fold higher as compared to normal oxygen tension was attributed to hypoxia induction of HIF-1 dependent growth factors such as VEGF, which are potent angiogenic stimuli when released under controlled conditions. Accordingly, culture of cells to be used for treatment of back pain may be performed in conditions of oxygen ranging from 0.5% to 4%, more preferably 1%- 3% and even more preferably from 1.5%- 1.9%. Hypoxia culture is not limited towards lowering oxygen tension but may also include administration of molecules that inhibit oxygen uptake or compete with oxygen uptake during the tissue culture process. Additionally, in an embodiment of the invention, hypoxia is induced through induction of agents that cause the upregulation of the HIF-1 transcription factor.

Subsequent to various culture procedures, cells generated may be tested for angiogenic and/or anti-inflammatory activity before use in clinical conditions. Testing may be performed by various means known to one skilled in the art. In terms of assessing angiogenic potential said means include, but are not limited to: a) Ability to support endothelial cell proliferation in vitro using human umbilical vein endothelial cells or other endothelial populations. Assessment of proliferation may be performed using tritiated thymidine incorporation or by visually counted said proliferating endothelial cells. A viability dye such as MTT or other commercially available indicators may be used; b) Ability to support cord formation in subcutaneously implanted matrices. Said matrices, which may include Matrigel or fibrin gel, are loaded with cells generated as described above and implanted subcutaneously in an animal. Said animal may be an immunodeficient mouse such as a SCID or nude mouse in order to negate immunological differences. Subsequent to implantation formation of endothelial cords may be assessed visually by microscopy. In order to distinguish cells stimulating angiogenesis versus host cells responding to said cells stimulating angiogenesis, a species-specific marker may be used; c) Ability to accelerate angiogenesis occurring in the embryonic chicken chorioallantoic membrane assay. Cells may be implanted directly, or via a matrix, into the chicken chorioallantoic membrane on embryonic day 9 and cultured for a period of approximately 2 days. Visualization of angiogenesis may be performed using in vivo microscopy; and d) Ability to stimulate neoangiogenesis in the hind limb ischemia model described above.

For all embodiments of the invention disclosed herein, cells to be used for treatment of lower back pain may be cryopreserved for subsequent use, as well as for transportation. One skilled in the art knows numerous methods of cellular cryopreservation. Typically, cells are treated to a cryoprotection process, then stored frozen until needed. Once needed cells require specialized care for revival and washing to clear cryopreservative agents that may have detrimental effects on cellular function. Generally, cryopreservation requires attention be paid to three main concepts, these are: 1) The cryoprotective agent, 2) the control of the freezing rate, and 3) The temperature at which the cells will be stored at. Cryoprotective agents are well known to one skilled in the are and can include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, or choline chloride as described in U.S. Pat. No. 6,461,645. A method for cryopreservation of cells, that is preferred by some skilled artisans is DMSO at a concentration not being immediately cytotoxic to cells, under conditions which allow it to freely permeate the cell whose freezing is desired and to protect intracellular organelles by combining with water and prevent cellular damage induced from ice crystal formation. Addition of plasma at concentrations between 20-25% by volume can augment the protective effect of DMSO. After addition of DMSO, cells should be kept at temperatures below 4 C, in order to prevent DMSO mediated damage. Methods of actually inducing the cells in a state of suspended animation involve utilization of various cooling protocols. While cell type, freezing reagent, and concentration of cells are important variables in determining methods of cooling, it is generally accepted that a controlled, steady rate of cooling is optimal. There are numerous devices and apparatuses known in the field that are capable of reducing temperatures of cells for optimal cryopreservations. One such apparatus is the Thermo Electro Cryomed Freezer [symbol] manufactured by Thermo Electron Corporation. Cells can also be frozen in CryoCyte [symbol] containers as made by Baxter. One example of cryopreservation is as follows: 2×106 CD34 cells/ml are isolated from cord blood using the Isolex System [symbol] as per manufacturer's instructions (Baxter). Cells are incubated in DMEM media with 10% DMSO and 20% plasma. Cooling is performed at 1 Celsius./minute from 0 to −80 Celsius. When cells are needed for use, they are thawed rapidly in a water bath maintained at 37 Celsius water bath and chilled immediately upon thawing. Cells are rapidly washed, either a buffer solution, or a solution containing a growth factor. Purified cells can then be used for expansion if needed. A database of stored cell information (such as donor, cell origination types, cell markers, etc.) can also be prepared, if desired.

1. A method of treating lumbar ischemia associated disc degenerative disease comprising administration of a therapeutic cell with enhanced retention ability, wherein said enhanced retention ability allows localization and mediation of therapeutic activities to ischemic areas.

2. The method of claim 1, wherein said therapeutic cell is an inducible pluripotent stem cell.

3. The method of claim 1, wherein said therapeutic cell is a mesenchymal stem cell derived from said inducible pluripotent stem cell.

4. The method of claim 3, wherein said mesenchymal stem cell possesses some features of mesenchymal stem cells but is not a mesenchymal stem cell.

5. The method of claim 4, wherein said cell possesses features of mesenchymal stem cells and features of endothelial cells.

6. The method of claim 4, wherein said cell possesses features of mesenchymal stem cells and features of nucleus pulposus cells.

7. The method of claim 4, wherein said cell possesses features allowing for enhanced angiogenesis.

8. The method of claim 7, wherein said cell is generated to produce enhanced levels of HGF as compared to non-manipulated cells.

9. The method of claim 7, wherein said cell is generated to produce enhanced levels of SERP-1 as compared to non-manipulated cells.

10. The method of claim 7, wherein enhanced angiogenic properties of said cells are endowed by transfection with one or more angiogenesis stimulating genes.

11. The method of claim 1, wherein said therapeutic cell is administered at a sufficient oxygen tension and for a sufficient time period to enhance stimulation production of FGF-1 at a level of more than 2 ng/ml per 1 million cells.

12. The method of claim 7, wherein enhanced angiogenic properties of said cells are endowed by exposure to valproic acid.

13. The method of claim 1, wherein said therapeutic cell is a pluripotent stem cell derived mesenchymal stem cell wherein said mesenchymal stem cell expresses interleukin-1 receptor antagonist upon stimulation with interferon gamma.

14. The method of claim 1, wherein said therapeutic cell is a pluripotent stem cell derived mesenchymal stem cell wherein said mesenchymal stem cell expresses interleukin-1 receptor antagonist upon stimulation with TRANCE.

15. The method of claim 1, wherein said therapeutic cell is a mesenchymal stem cell engineered to express a chimeric antigen receptor.

16. The method of claim 15, wherein said chimeric antigen receptor comprises of an immunoglobulin domain and a T cell receptor signaling domain.

17. The method of claim 15, wherein said cell engineered to express said CAR is also modified to possess enhanced proclivity towards hypoxia.

18. The method of claim 17, wherein said proclivity towards hypoxia is mediated by augmented expression of receptors mediating chemotaxis towards messengers released by hypoxic cells.

19. The method of claim 18, wherein said messenger released by said hypoxic cells is kisspeptin.

20. The method of claim 18, wherein said messenger released by said hypoxic cells is vasoactive intestinal peptide.
